(19) United States
(12) Patent Application Publication
(10) Pub. No.: US 2013/0158532 A1
(43) Pub. Date: Jun. 20, 2013

(54) LASER CATHETER FOR BYPASS SURGERY

(71) Applicant: Cornelis Antonius Franciscus Tulleken, Utrecht (NL)

(72) Inventor: Cornelis Antonius Franciscus Tulleken, Utrecht (NL)

(21) Appl. No.: 13/805,296

(22) PCT Filed: Jun. 24, 2010
(86) PCT No.: PCT/NL2010/050395

§ 371 Date: Mar. 4, 2013

(51) Int. Cl. A61B 18/24

(52) U.S. Cl. CPC A61B 18/24
USPC 606/15

(57) ABSTRACT

The invention concerns an assembly for by-pass surgery. The assembly comprises a laser catheter. The laser catheter comprises: a tubular arrangement of optical fibres having distal ends defining a ring-shaped emitting surface arranged for emitting a tubular bundle of laser beams in a distal direction of the catheter; and a gripper for gripping tissue inside the tubular bundle of laser beams, having a gripping end defining the location where gripped tissue is held, the gripping end being arranged inside the tubular bundle and proximally from the emitting surface. The distance between the emitting surface and the gripping end is at least 1 mm.

### FIELD OF THE INVENTION

The present invention relates to an assembly for by-pass surgery comprising a laser catheter, which laser catheter comprises: a tubular arrangement of optical fibres having distal ends defining a ring-shaped emitting surface arranged for emitting a tubular bundle of laser beams in a distal direction of the catheter; and a gripper for gripping tissue inside the tubular bundle of laser beams, having a gripping end defining the location where gripped tissue is held, the gripping end being arranged inside the tubular bundle and proximally from the emitting surface.

### BACKGROUND OF THE INVENTION

An assembly for by-pass surgery comprising a laser catheter is known from EP 750,476. This document describes the use of such an assembly in the ELANA® (Excimer Laser Assisted Non-occlusive Anastomosis) operating technique. For this technique, one requires a catheter and a ring, which are jointly called Elena® Arteriotomy System.

The catheter disclosed in EP 750,476 is used for performing an ETS-anastomosis (ETS=End To Side) between a graft vessel and a target vessel. The graft is fixed with an end to the side of the target vessel, while the blood flow through the target vessel, also called recipient vessel, is not interrupted, i.e. blood continues to flow through the target vessel while performing the anastomosis. For this purpose, first the graft vessel is fixed to the target vessel and subsequently, after this fixation is established, the flow connection between the target vessel and graft vessel is made by removing the part of the wall of the target vessel which lies in front of the fixed end of the graft vessel. Said part of the wall of the target vessel is removed by means of a tubular arrangement of optical fibres emitting a tubular bundle of laser beams originating from the fibres and a suction gripper provided inside the tubular arrangement of optical fibres. The tubular bundle of laser beams burns a ring shaped cut into the wall of the target vessel, resulting in a ring-based passage connecting the lumens of the graft vessel and target vessel. The ring-based wall part of the target vessel—i.e. the part lying inside said burned circle, which part is also called the “flap”—is gripped by the suction gripper and removed together with the withdrawal of the catheter after the burning operation.

When cutting, the laser catheter burns a ring shaped groove in the wall of the target vessel. The depth of this groove becomes deeper and deeper, until the depth of the groove amounts to the wall thickness of the target vessel and the so called flap is normally set free from the surrounding wall of the graft vessel. Subsequently, the flap can be removed by means of the gripper. This technique performs very well and reliable on target vessel with a wall thickness up to, say 0.5-0.7 mm. However, when applying this technique to target vessels with relatively thick walls—say 1.0 mm and more—removing the flap encounters difficulties. Taking into account that it is of great importance that the so called flap is removed completely and reliably, additional actions must be taken in order to ensure that the flap is removed completely. Otherwise, the flap or parts of it might obstruct the blood flow.

### SUMMARY OF THE INVENTION

The object of the present invention is to provide an improved assembly for bypass surgery overcoming among others the above problem associated with relatively thicker target vessels.

This object is achieved by providing an assembly for by-pass surgery comprising a laser catheter, which laser catheter comprises:
a tubular arrangement of optical fibres having distal ends defining a ring-shaped emitting surface arranged for emitting a tubular bundle of laser beams in a distal direction of the catheter;
a gripper for gripping tissue inside the tubular bundle of laser beams, having a gripping end defining the location where gripped tissue is held, the gripping end being arranged inside the tubular bundle and proximally from the emitting surface,
   wherein the assembly is characterized in that the distance between the emitting surface and the gripping end is at least 1 mm.

An advantage of such a distance between the emitting surface and the gripping end, is that the tubular arrangement of optical fibres can be inserted into the wall of the vessel during the cutting, with reduced pressing of the gripper end onto the target vessel's wall, which could cause the target vessel to be dislocated. A further advantage is that emitting surface can follow the instantaneous burning surface closer whilst the depth of the ring shaped groove increases during burning. It appears that the smaller the distance between the emitting surface and the instantaneous burning surface onto which the laser beams impinge, the better the burning action. It is believed that the explanation for this might be that burning causes gasses which reduce the effectiveness of the laser beams, i.e., the effectiveness of the burning action.

According to a further embodiment of the assembly according to the invention, said distance between the emitting surface and the gripping end is at least 2 mm. Taking into account the wall thickness of relatively thick target vessels onto which bypass surgery is in practise performed, this minimum distance appears to provide reliable cuts for the most commonly occurring relatively thick target vessels.

According to a further embodiment of the assembly according to the invention, said distance between the emitting surface and the gripping end is at most 3.5 mm, preferably at most 3.0 mm. When the distance becomes too large, the gripper end might not reliably grip the so called ‘flap’ surrounded by the groove cut by the laser beams.

According to a further embodiment of the assembly according to the invention, said distance between the emitting surface and the gripping end is in the range of 2-3 mm, preferably about 2.5 mm. Taking into account, on the one hand, the thickness of the wall of generally occurring target vessels and, on the other hand, a reliable gripping of the so called flap, this range for the distance between the emitting surface and the gripping end appears to be about optimal for many, if not most, of bypass surgery procedures on thicker target vessels.

According to a further embodiment of the assembly according to the invention, the gripping end is defined by a grid at the distal end of the channel, which grid preferably extends across the channel. More preferably the grid is parallel to the emitting surface. The grid prevents the so called flap from entering deeply into the catheter, so that after removal of the catheter from the graft vessel, visual inspection will easily show whether or not the flap is removed. In case the flap might not be removed further actions are necessary in order to remove the flap.

According to a further embodiment of the assembly according to the invention, the assembly further comprises a stop surface extending around the bundle of optical fibres and facing in the distal direction of the catheter, wherein the stop surface is arranged at a distance proximally from the emitting surface. The stop surface prevents the tubular arrangement of optical fibres from advancing too far into the target vessel. This because the stop surface has a larger diameter than the whole burned into the wall of the target vessel. Preferably the stop surface is arranged proximally from the gripping end. This in order to ensure that the emitting surface can follow the burning surface onto which the laser beams impinge as close as possible. Viewed in the longitudinal direction of the laser catheter, the distance from the gripper end to the stop surface is in the range of 0.35-0.85 mm, preferably about 0.5 mm. When the distance from the gripping end to the stop surface is in the range of 0.35-0.85 mm, it will allow the tubular arrangement to be inserted into the tubular groove until the wall of the target vessel is completely cut through.

According to a further embodiment, the assembly according to the invention further comprises a graft vessel to be mounted end-to-side on the sidewall of a target vessel of a patient, the graft vessel having an inner diameter; wherein the laser catheter has an external diameter defined by the outer wall of the catheter, having an external outer wall diameter; and wherein the inner diameter of the graft vessel is at least 0.1 mm larger than said external diameter of the laser catheter. The inner diameter of the graft vessel being at least 0.1 mm or 0.2 mm larger than the external outer wall diameter of the laser catheter, reduces friction between the outer wall of the laser catheter and the inner wall of the graft vessel to such an extent that the laser catheter can smoothly advance through the graft vessel during cutting. This allows the distal ends of the optical fibres to follow the bottom of the groove closely, when the depth of the groove becomes deeper and deeper during cutting. The distal ends of the optical fibres advancing into and in the groove towards the bottom of the groove, not only enables complete and reliable removal of the flap with larger wall thickness—up to 3 mm and more—but also improves the technique when applied to target vessels with relatively thin vessel walls—say up to 0.5-1.0 mm. A possible explanation might be that gasses generated due to burning away tissue and present between the distal ends of the optical fibres and the bottom of the groove, reduce the effectiveness of the laser beams. This might suggest that one should keep the distal ends of the optical fibres at some distance to the groove in order to allow the gas to escape, as the optical fibres close off the upper side of the groove. However, surprisingly, it proved to be very effective to keep said distal ends close to the bottom of the groove.

In another embodiment according to the invention, the inner diameter of the graft vessel is at most 0.5 mm larger than said external diameter of the graft vessel and/or the inner diameter of the graft vessel is preferably at least 0.3 mm larger than said external diameter of the catheter. Yet in another embodiment the external diameter of the catheter is at least 1.8 mm, preferably at least 2.0 mm.

When the play between the inner wall of the graft vessel and the outer wall of the laser catheter becomes too large, the passage burnt into the wall of the target vessel becomes too small relative to the inner diameter of the graft vessel. This is disadvantageous for the flow properties of blood flowing through the graft vessel.

In another embodiment the assembly further comprises a ring member having dimensions adapted for, on the one hand, insertion of the distal end of the tubular arrangement of optical fibres through said ring member and for, on the other hand, preventing passage of the stop surface through said ring member. In a further embodiment, the outer diameter of the grafts vessel allows insertion of the graft vessel through said ring member.

Before connecting the graft vessel to the target vessel, the graft vessel will be prepared for the bypass procedure by inserting one end of the graft vessel through the ring member. Before using the laser catheter, this end of the graft vessel will be attached to the wall of the target vessel. Subsequently, when the laser catheter has been introduced into the graft vessel and the laser operation is performed, the ring member will prevent the laser catheter from advancing too far into the target vessel as soon as the stop surface comes to rest onto the ring member. Note however, that the laser catheter can also be inserted into the graft vessel fully outside the patient, i.e. when the graft vessel is separate (not connected to) the patient. Therefore, according to a further embodiment the laser catheter is positioned inside the graft vessel whilst the graft vessel is separate from the patient.

### BRIEF DESCRIPTION OF THE DRAWINGS

Further advantageous embodiments of the assembly according to the invention are described in the claims and in the following description with reference to the drawing, in which:

FIG. 1 shows a laser catheter of an assembly known in the art, wherein FIG. 1A is a longitudinal view in cross section and FIG. 1B shows an end view according to arrow lb in FIG. 1A.

FIG. 2 shows an assembly according to the invention and a sequence of steps in an ETS-anastomosis procedure according to the invention, wherein FIG. 2 is sub-divided into the FIGS. 2a, 2b, 2c and 2d, which each show a different step.

FIG. 3 shows the assembly according to the invention, with a laser catheter inside a graft vessel attached to a thick wall of a thick vessel, while cutting.

### DETAILED DESCRIPTION OF THE INVENTION

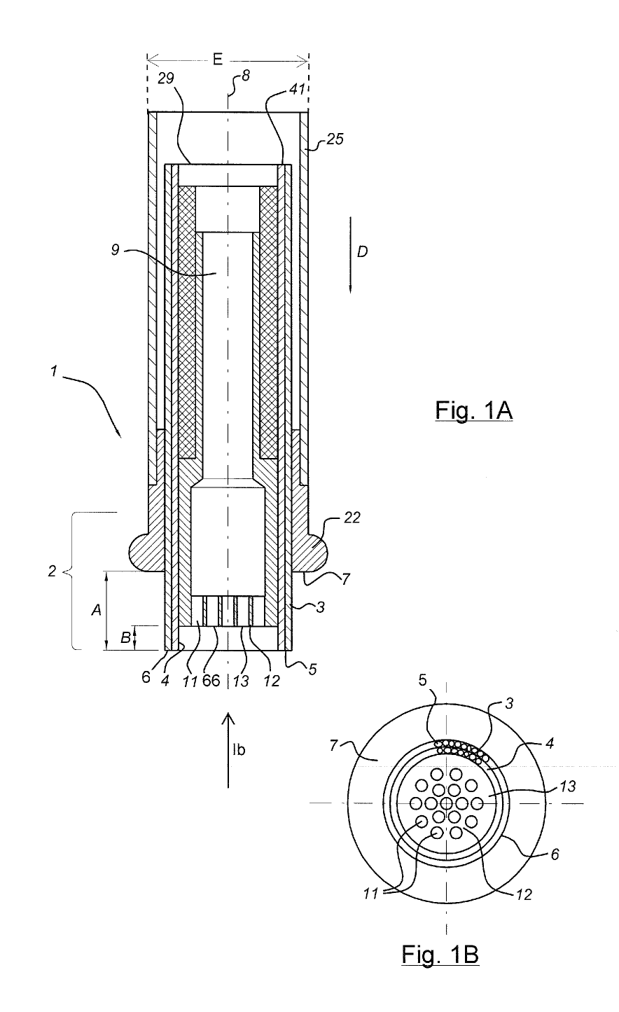

FIG. 1 shows a laser catheter 1 of an assembly according to the invention. The distal part 2 of the laser catheter 1 is provided with a tubular arrangement 3 of optical fibres 4. The optical fibres 4 have distal ends 5, which together define a ring-shaped emitting surface 6. When a laser source is connected to the proximal ends 41 of the optical fibres 4, a laser beam will emit from each of these distal ends 5 of the optical fibres 4. The distal ends of the optical fibres 4 extend parallel to the longitudinal axis 8 of the catheter, so that the emitted laser beams will extend parallel to the longitudinal axis 8 in the distal direction indicated by arrow D. This results in a tubular bundle of laser beams in the distal direction D of the catheter.

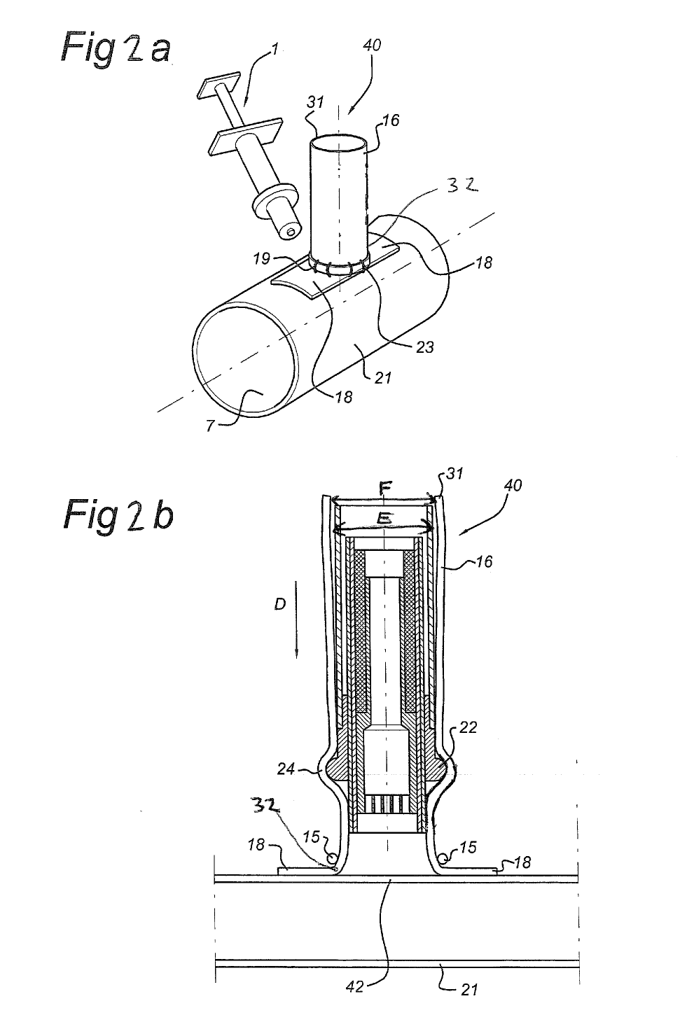

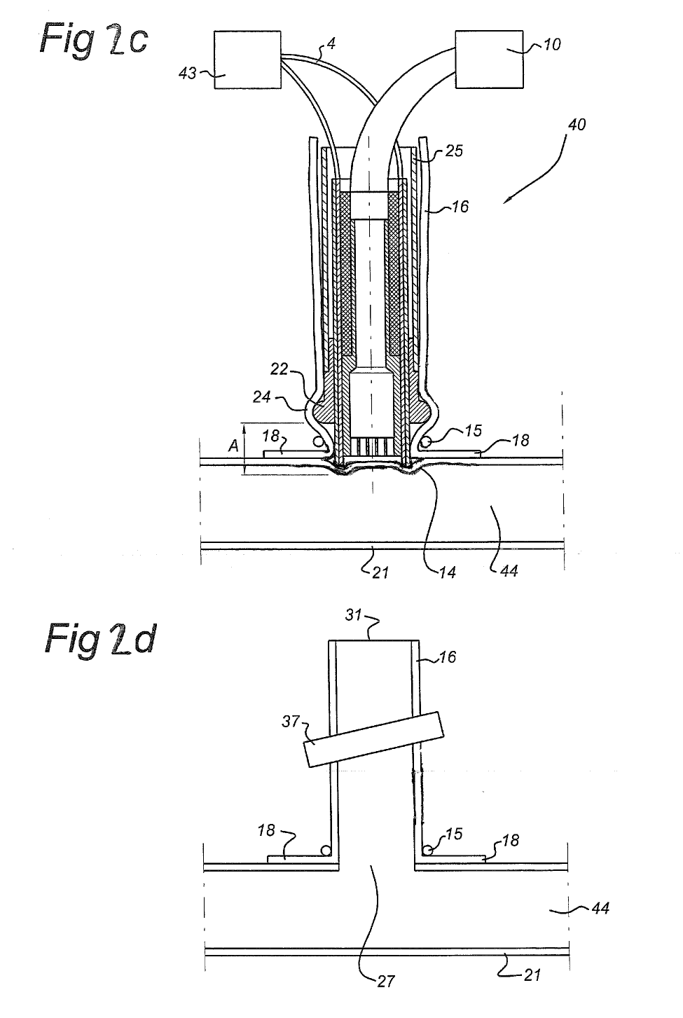

The laser catheter 1 further comprises a casing 25 surrounding the tubular arrangement 3 of optical fibres. In FIG. 1a, the outer diameter of casing 25 is the external outer wall diameter of the laser catheter, indicated by arrow E. The tubular arrangement 3 of optical fibres 4 encloses a channel 9. The proximal end of the channel 9 can be connected to a vacuum source 10 (see FIG. 2c) in order to apply a suction force to the channel 9. The distal end of the channel 9 is provided with a plate 66, defining the suction surface 13 and provided with suction apertures 11 (see FIG. 1b). This plate 66 is also referred to as grid 66. The distal end of the channel 9 thus forms a suction mouth 12, which acts as a gripper when vacuum is applied at the proximal end of channel 9. The suction mouth 11 is provided at a distance B proximally from the emitting surface 6. This distance B will at least be about the thickness of the wall of the target vessel (see 21 in FIG. 2).

The distal end of the casing 25 is provided with a radial rib 22. The distal side of the radial rib 22 forms a stop surface 7. This stop surface lies proximally at a distance A from the emitting surface 6.

In order to ensure a good gripping of the flap 14 (FIG. 2c)—the term ‘flap’ indicates ‘the tissue part separated after cutting away the ring of tissue—by the suction mouth 12, the suction surface 13 of the suction mouth extends parallel to the emitting surface 6.

The rib 22 with the stop surface 7 also extends parallel to the emitting surface 6. It will be clear that the rib 22 is preferably a rib extending continuously around the catheter, but that it may also be a discontinuous rib. The outer diameter of the rib 22 is larger than the inner diameter of the ring member 15. This reliably prevents the distal part of the catheter from being inserted too far into the target vessel.

Referring to FIG. 1B, it is noted that only a few distal end of optical fibres are actually shown. Only two concentric parts (each part about ⅛ of a circle large) of two circles of distal ends 5 of optical fibres are shown. It will however be clear that each ‘⅛ circle part’ will actually extend all around over 360°.

Further referring to FIG. 1B, it can be seen that the distal ends 5 of the optical fibres 4 lie closely packed together with the longitudinal walls of adjacent fibres against each other to form together a tubular arrangement 3 having a ring-based cross-section as can be seen in FIG. 1B. The distal end faces of all the optical fibres 4 together define an essentially flat emitting surface 6, which extends perpendicular to the longitudinal axis 8 of the catheter. Due to the distal ends 5 of the optical fibres being closely packed, the bundle of laser beams, which are emitted when a laser source is connected, form an essentially continuous bundle which is capable of burning away a continuous ring of tissue from a target vessel.

The distal ends of the optical fibres being closely packed can also be elucidated as follows: Taking into account that the distal ends 5 of adjacent optical fibres lie against each other and that in this manner a closed ring of distal ends 5 of optical fibres is obtained—which closed ring is an essentially continuous ring—, the laser beams emitting from the distal ends of the fibres will—in case all fibres emit simultaneously a laser beam—together form a compound tubular laser beam, which is in circumferential direction of the compound tubular laser beam essentially continuous. Note however that, the fibres might also emit laser beams sequentially instead of simultaneously. In both cases a ring shaped groove can be burnt into the wall of the target vessel. As can be seen in FIG. 1b the tubular bundle of optical fibres might comprise two concentric rings of fibres. Note however, that also more concentric rings of optical fibres might be provided.

Referring to FIGS. 2a-2d, an ETS-anastomosis procedure with the above catheter will be described.

FIG. 2a shows a first step. The distal end 32 of the graft vessel 16 is attached to the side wall of the target vessel 21, leaving the part 42 (see FIG. 2b) of the wall tissue of the target vessel 21 in front of the lumen of the graft vessel 16 intact so that the blood flow in the target vessel 21 can be left undisturbed as there is no leakage possible. The graft vessel 16 can be fixed to the target vessel 21 by any suitable connection technique, such as connection techniques known from the prior art which preferably do not require the part 42 of wall tissue to be removed before. FIG. 2a shows for example a suture 23 enclosing the ring member 15 as well as piercing through the graft vessel 16 and the target vessel 21. Instead of a suture 23 also a staple could be used. Further, as is shown in FIG. 2, the flanges 18 can be used for establishing a good connection to the target vessel 21. The flanges 18 can for example be glued to the target vessel 21.

After a firm and sufficiently leak tight connection between the graft vessel 16 and target vessel 21 has been established, the laser catheter 1 of FIG. 1 is inserted into the proximal end 31 of the graft vessel 16, see FIG. 2b. As can be seen in FIG. 2b, the rib 22 on the outer circumference of the laser catheter 1 causes a similar rib 24 in the wall of the graft vessel 16. This rib 24 allows the surgeon to see how far the catheter is advanced in the graft vessel 16.

The laser catheter 1 is advanced distally (arrow D in FIG. 2b) up to the emitting surface 6 contacts the wall part 42 to be removed from the target vessel. In case not already done before, the channel 9 and optical fibres 4 are, subsequently, connected to a vacuum source 10 and laser source 43, respectively. A vacuum is applied to the channel 9 and the laser procedure is started. Laser radiation is emitted into the optical fibres 4 and the so called flap 14 is cut. The flap 14 is gripped by the suction mouth 11. At this moment, the laser procedure is finished and the laser source can be switched off. Subsequently, the laser catheter is retracted in the direction opposite to arrow D, whilst the flap 14 is being removed by the suction gripper 11.

As soon as the laser catheter has been retracted over a sufficient distance, a clip 37 (FIG. 4d) or other closure is placed on the graft vessel 16 in order to close it off. Blood will be allowed to enter the graft vessel through the aperture 27, but will not be able to pass the clip 37. The proximal end 31 of the graft vessel can be connected by a ETE-anastomosis (ETE=End To End) to another vessel, such as an other graft vessel, or it can be connected by an ETS-anastomosis to the same or another target vessel.

The laser catheter and its use as described up to here in the ‘detailed description’ corresponds essentially to the laser catheter disclosed in EP 750,476. Next, more specifically the present invention will be addressed.

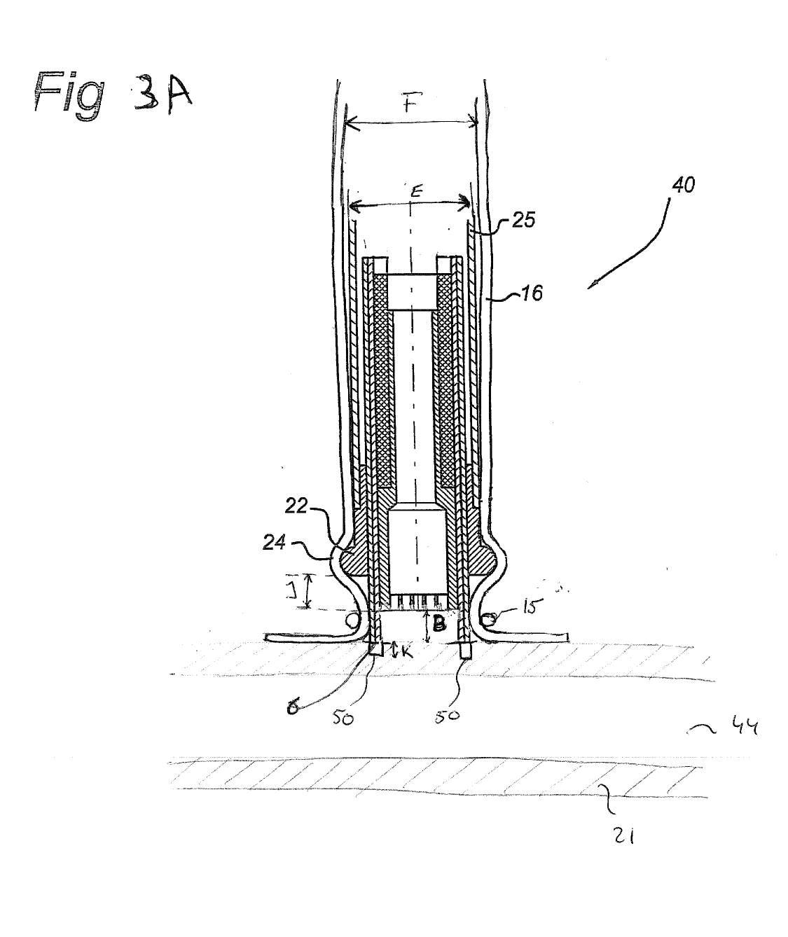

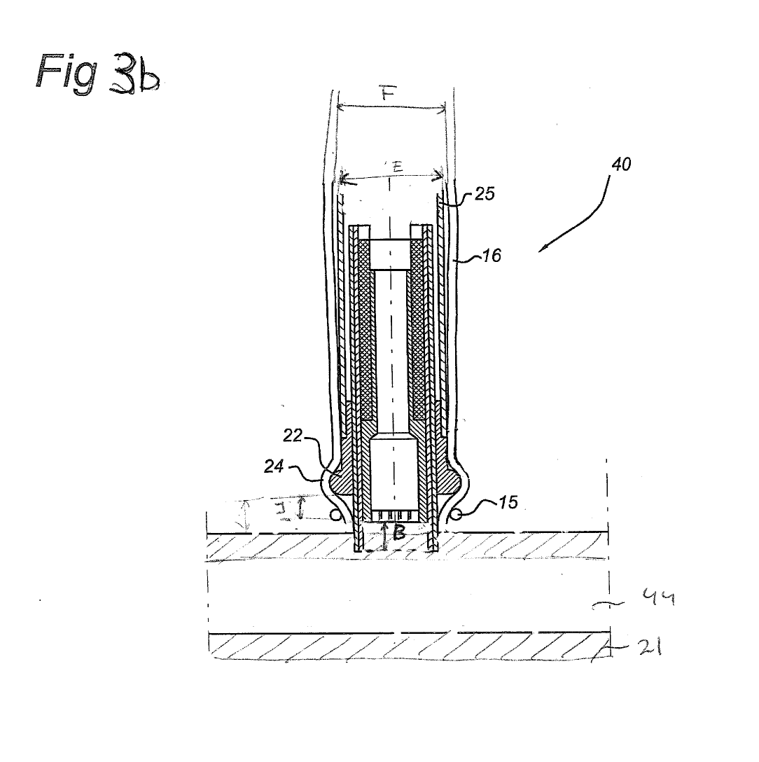

FIG. 3 shows the assembly according to the invention while cutting the target vessel wall. FIG. 3b shows situation a short time after the situation of FIG. 3a.

In the beginning of the cutting process, the laser beams have cut a ring shaped groove 50 in the wall of the target vessel. The groove 50 has a depth indicated very schematically by arrow K in FIG. 3a. After cutting groove 50, the tubular arrangement of optical fibres advances into the groove 50, as can been seen in FIG. 3b. In this way, the emitting surface 6 can closely follow the bottom of the groove 50. Although it is described above as a two-step process, i.e. first cutting the groove 50 and secondly inserting the tubular arrangement of fibres, in practice this is a continuous process. While the groove 51 is being deepened by the cutting, the tubular arrangement of optical fibres is being advanced in the groove.

During this process the laser catheter 1 is moved towards the centre axes of the target vessel. According to the invention, inner wall diameter F of the graft vessel is larger than outer wall diameter of the laser catheter E and therefore, friction between the outer wall of the laser catheter and the inner wall of the graft vessel is limited. Friction essentially only remains in the region of the rib 22, which serves as a stop preventing the distal end of the laser catheter from being inserted too far into the target vessel 44.

For a complete cutting it is advantageous that the emitting surface can be advanced completely through the target vessel wall. From FIG. 3 it can be seen that the proximal distance between the ring-shaped emitting surface and the gripping surface, indicated by arrow B, should be at least equal to the thickness of the target vessel. As can also be seen from FIG. 3, the tubular arrangement of optical fibres can only be inserted in target vessel wall until the rib comes to rest onto the vessel wall or, when applied, onto the ring member. The stop surface, defined by the distal side of the rib is arranged proximally from the gripping surface is indicated by arrow J.

The assembly further comprises a ring member 15. The size of the ring member is such that the distal end of the tubular arrangement of optical fibres can be inserted through the ring member, while the passage of the stop surface through the ring member is prevented. In an embodiment of the invention, the distance from the gripper surface to the stop surface, indicated by arrow J, is in the range of 0.35-0.85 mm, preferably about 0.5 mm. The ring member will prevent the laser catheter from advancing too far into the target vessel as soon as the stop surface comes to rest onto the ring member. At that point the suction surface is positioned again the target vessel wall and will be able to remove the ring-based wall part of the target vessel.

The next following clauses define a second invention different from the invention as defined by the claims. This second invention might later form the basis for a divisional application. The clauses are:
1) Assembly for by-pass surgery comprising a laser catheter, which comprises a tubular arrangement of optical fibres having distal ends defining a ring-shaped emitting surface for emitting a tubular bundle of laser beams in a distal direction of the catheter, the laser catheter being defined by an essential tubular, having an external outer wall diameter; and a graft vessel to be mounted end-to-side on the sidewall of a target vessel of a patient, the graft vessel having an inner diameter, characterized in that the inner diameter of the graft vessel is at least 0.1 mm larger than said external outer wall diameter.
2) Assembly according to clause 1, wherein the inner diameter of the graft vessel is at least 0.2 mm larger than said external outer wall diameter.
3) Assembly according to one of clauses 1-2, wherein the inner diameter of the graft vessel is at most 0.5 mm larger than said external outer wall diameter; and/or wherein the inner diameter of the graft vessel is at least 0.3 mm larger than said external outer wall diameter.
4) Assembly according to one of clauses 1-3, wherein the external outer wall diameter is at least 1.8 mm, preferably at least 2.0 mm.
5) Assembly according to one of clauses 1-4, wherein the external outer wall diameter is in the range of 2.0-2.2 mm.
6) Assembly according to one of clauses 1-5, wherein the laser catheter further comprises a gripper for gripping tissue inside the tubular bundle of laser beams, having a distal end defining a gripping surface, the gripping surface being arranged parallel to the emitting surface and at a proximal distance from the emitting surface, characterized in that the proximal distance between the ring-shaped emitting surface and the gripping surface is at least 1 mm.
7) Assembly according to clause 6, wherein said proximal distance is at least 2 mm.
8) Assembly according to one of clauses 6-7, wherein said proximal distance is at most 3.5 mm, preferably at most 3.0 mm.
9) Assembly according to one of clauses 6-8, wherein said proximal distance is in the range of 2-3 mm, preferably about 2.5 mm.
10) Assembly according to one of clauses 1-9, wherein the cylindrical outer wall carries at its distal end a circumferential rib, having an outside rib diameter larger than said external outer wall diameter;
11) Assembly according to clause 10, wherein the distal side of the rib defines a stop surface extending around a tubular arrangement of optical fibres; and wherein the stop surface is arranged proximally from the gripping surface at a distance from the emitting surface.
12) Assembly according to clause 11, wherein viewed in the longitudinal direction of the laser catheter, the distance from the gripper surface to the stop surface is in the range of 0.35-0.85 mm, preferably about 0.5 mm.
13) Assembly according to one of clauses 11-12, further comprising a ring member having dimensions adapted for, on the one hand, insertion of the distal end of the tubular arrangement of optical fibres through said ring member and for, on the other hand, preventing passage of the stop surface through said ring member.
14) Assembly according to clause 13 in dependence on one of the clauses 8-12, wherein the outer diameter of the grafts vessel allows insertion of the graft vessel through said ring member.
15) Assembly according to one of clauses 1-14, wherein the laser catheter is positioned inside in the graft vessel.

Referring to the preceding explanation of the invention, it is noted that further modifications and embodiments are very well conceivable. Also further modifications and embodiments are within the scope of this invention.

1. Assembly for by-pass surgery on a target vessel having a vessel wall comprising a laser catheter, which laser catheter comprises:
a tubular arrangement of optical fibres having distal ends defining a ring-shaped emitting surface arranged for emitting a tubular bundle of laser beams in a distal direction of the catheter;
a gripper for gripping tissue inside the tubular bundle of laser beams, having a gripping end defining the location where gripped tissue is held, the gripping end being arranged inside the tubular bundle and proximally from the emitting surface,
characterized in that
the distance between the emitting surface and the gripping end is equivalent to a thickness of the wall of the target vessel.

2. Assembly according to claim 1, wherein said distance between the emitting surface and the gripping end is at least 2 mm.

3. Assembly according to claim 1, wherein said distance between the emitting surface and the gripping end is at most 3.5 mm, preferably at most 3.0 mm.

4. Assembly according to claim 1, wherein said distance between the emitting surface and the gripping end is 2.5 mm.

5. Assembly according to claim 1, wherein the gripper comprises a hollow channel extending within the tubular arrangement and connectable to a vacuum source.

6. Assembly according to claim 5, wherein the assembly further comprises a vacuum source connected to said channel.

7. Assembly according to claim 5, wherein the gripping end is defined by a grid at the distal end of the channel, which grid preferably extends across the channel.

8. Assembly according to claim 1, wherein the assembly further comprises a stop surface extending around the bundle of optical fibres and facing in the
distal direction of the catheter, wherein the stop surface is arranged at a distance proximally from the emitting surface.

9. Assembly according to claim 8, wherein the stop surface is formed by a circumferential rib provided on the outer wall of the catheter, and wherein the rib has an outside rib diameter larger than the external diameter of the outer wall of the catheter.

10. Assembly according to claim 8, wherein the stop surface is arranged
proximally from the gripping end.

11. Assembly according to claim 10, wherein, viewed in the longitudinal direction of the laser catheter, the distance from the gripping end to the stop surface is in the range of 0.35-0.85 mm, preferably about 0.5 mm.

12. Assembly according to claim 1, wherein the target vessel is a graft vessel to be mounted end-to-side on the sidewall of a target vessel of a patient, the graft vessel
having an inner diameter;
wherein the laser catheter has an external diameter defined by the outer wall of the catheter, having an external outer wall diameter; and
wherein the inner diameter of the graft vessel is at least 0.1 mm larger than said external diameter of the laser catheter.

13. Assembly according to claim 12, wherein the inner diameter of the graft vessel is at least 0.2 mm larger than said external diameter of the catheter.

14. Assembly according to claim 12, wherein the inner diameter of the graft vessel is at most 0.5 mm larger than said external diameter of the catheter;
and/or
wherein the inner diameter of the graft vessel is at least 0.3 mm larger than said external diameter of the catheter

15. Assembly according to one of claim 12, wherein the external diameter of the catheter is at least 1.8 mm, preferably at least 2.0 mm.

16. Assembly according to claim 12, wherein the external diameter of the catheter is in the range of 2.0-2.2 mm.

17. Assembly according to claim 1, wherein the assembly further comprises a ring member having dimensions adapted for, on the one hand, insertion of the distal end of the tubular arrangement of optical fibres through said ring member and for, on
the other hand, preventing passage of the stop surface through said ring member.

18. Assembly according to claim 17, wherein the outer diameter of the grafts vessel allows insertion of the graft vessel through said ring member.

19. Assembly according to claim 12, wherein the laser catheter is positioned inside in the graft vessel.

20. Assembly according to claim 1, wherein the laser catheter is arranged to provide laser radiation having a wavelength in the range of 100-500 nm.

21. Assembly according to claim 1, wherein the laser catheter is an excimer laser catheter.

22. Assembly according to claim 1, wherein the distal ends of
adjacent optical fibres lie against each other so that a closed ring of distal ends of optical fibres is obtained.
